(19) United States
(12) Patent Application Publication
(10) Pub. No.: US 2023/0207076 A1
(43) Pub. Date: Jun. 29, 2023

(54) CLINICAL DATA CONNECTOR AND CLINICAL DOCUMENT COLLECTOR FOR INDIVIDUAL ACCESS

(71) Applicant: Change Healthcare Holdings, LLC, Nashville, TN (US)

(72) Inventors: Genevieve Lynn Morris, Pooler, GA (US); Peter Bernhardt, Albany, CA (US); Stephen Dean, Henrico, NC (US); Rashmi Jagannath, Fremont, CA (US); Faisal Mushtaq, West Harrison, NY (US)

(21) Appl. No.: 17/558,288

(22) Filed: Dec. 21, 2021

(51) Int. Cl. G16H 10/60; G06F 40/106; G06F 40/205

(52) U.S. Cl. CPC G16H 10/60; G06F 40/106; G06F 40/205

(57) ABSTRACT

A system and method are described for collecting and providing health-related information of a patient’s medical record or information from a plurality of health-service providers using a clinical data connector and clinical document collector. In some instances, all or a part of the collected information are received through a web application or web service (e.g., of a third-party requestor).

### BACKGROUND

Patients make purchases of health-related services all the time, e.g., to purchase life insurance, disability insurance, or supplemental or secondary life insurance. Patients also file claims, e.g., for related injuries or automotive-related injuries. To process these purchases or claims, third-party entities such as life insurers and secondary/supplemental insurance organizations, insurance companies, and various third parties require access to the patient’s medical history or records.

Currently, it is the responsibility of the patient to get the patient’s medical history or records to these third-party entities. Often, the patient would have to prepare and submit a HIPAA authorization form for each doctor or health-service provider that is required by the third-party entity. The process is time-consuming, particularly on the part of the patient, and it is often incomplete. Because the responsibility is on the patient to identify all of his or her healthcare service providers, omissions can occur unintentionally, e.g., if the patient omits information inadvertently or if the patient did not believe certain information to be pertinent. In certain instances, omissions can be intentional. Similarly, life insurers, secondary/supplemental insurance organizations, insurance companies, and various third parties, when assessing the patient health to sell a health-related service, desire a complete medical history of the patient.

Efforts are being made to streamline the exchange of patient medical records. There are companies that are providing services for the retrieval of patient medical records. The process is still highly manual. The company would receive multiple facsimiles with HIPAA authorization forms from a patient. The company then contacts the medical provider using the information provided in the form to retrieve the patient medical records. The company then manually collates records and transmit the facsimiles of the records to the requestors. Electronic versions of the same are available.

### SUMMARY

A system and method are described for collecting and providing health-related information of a patient’s medical record or information from a plurality of health-service providers using a clinical data connector and clinical document collector. In some instances, all or a part of the collected information is received through a web application or web service (e.g., of a third-party requestor). The web application or web service is configured to receive consent from the patient for their medical records, e.g., for a reason/purpose of use of the medical records. In some embodiments, the web application or web service is configured to present a general consent form to the patient through a customer portal curated by the web application or web service to receive the authorization from the patient. The web application or web service then performs a verification of the identity of the patient using the provided information and transmits a request having the proof of validation and patient information to the clinical data connector. In some embodiments, the web application or web service operates with other verification services.

The clinical data connector searches and matches all records related to the patient using the provided patient information to identify service providers. In some embodiments, the clinical data connector generates a task that is serviced by the clinical document collector to interrogate the service provider’s databases, through an API, to determine if it has medical records of the patient. The clinical document collector then collects the medical records from the service provider’s databases, e.g., through the API.

In this workflow, the patient does not have to identify or provide all of this or her medical provider. The clinical data connector and clinical document collector can find by accessing networks of data and performing a patient match. In addition, the patient does not necessarily have to complete an individual HIPAA authorization form for each requested information or for each provider. The general consent form, as provided through the web application or web service, can serve as a proxy for the HIPPA authorization form, or it can be used to generate the HIPPA authorization forms to be provided to each provider from which the medical records are being retrieved. In addition, the clinical data connector and clinical document collector can parse through the provided medical records to populate a specific set of requested medical information.

In an aspect, a system is disclosed comprising at least one computing device having instructions stored thereon that executes a clinical data connector, and a clinical document collector configured to (a) receive a request for clinical documents or clinical information of a patient, the request having (i) a plurality of patient identifier, (ii) a plurality of patient demographic identifier, and (iii) a purpose of use code for the request, (b) verify the request and match a patient in a database using (i) and (ii); (c) retrieve a plurality of clinical documents or clinical information; and (d) transmit the plurality of clinical documents or clinical information a requester or designated third party receiver. The clinical data connector is configured by instructions to execute a plurality of operations in an order to retrieve the plurality of clinical documents or clinical information, wherein each operation creates a task for the clinical document collector and receives a plurality of clinical documents or clinical information from the clinical document collector. The clinical document collector is configured by instructions to execute a plurality of operations in an order to retrieve the plurality of clinical documents or clinical information, wherein each operation receive the task from the clinical data connector; retrieves the plurality of clinical documents or clinical information from the plurality of databases; and transmits the plurality of clinical documents or clinical information to the clinical data connector.

In some embodiments, the request for the clinical documents or clinical information is received from a customer portal application, wherein the customer portal application is configured by instructions to present and receive, via a graphical user interface, an executed general consent form from the patient.

In some embodiments, the request comprises an executed general consent form from the patient.

In some embodiments, the request from the customer portal application is through a SMART on FHIR API.

In some embodiments, the clinical document collector is configured by instructions to search a health exchange system for an available patient access list, wherein the available patient access list is used to create the task for the clinical document collector.

In some embodiments, the clinical data connector is configured to execute a state machine to track the retrieval of the plurality of clinical documents or clinical information.

In some embodiments, the clinical data connector is configured to poll the clinical document collector for a status request of each task, and wherein the reply of the status request include a link to a document store.

In some embodiments, the clinical data connector is configured to request the clinical document collector to request for the clinical documents or clinical information of the patient from the document store by sending a document request the document store using the link.

In some embodiments, the clinical data connector is configured to receive the request to initiate the operation for the retrieval of the clinical documents or clinical information of the patient from a third-party website or portal.

In another aspect, a non-transitory computer-readable medium is disclosed having instructions stored thereon that executes a clinical data connector, and a clinical document collector configured to (a) receive a request for clinical documents or clinical information of a patient, the request having (i) a plurality of patient identifier, (ii) a plurality of patient demographic identifier, and (iii) a purpose of use code for the request, (b) verify the request and match a patient in a database using (i) and (ii); (c) retrieve a plurality of clinical documents or clinical information; and (d) transmit the plurality of clinical documents or clinical information a requester or designated third party receiver. The clinical data connector is configured by instructions to execute a plurality of operations in an order to retrieve the plurality of clinical documents or clinical information, wherein each operation creates a task for the clinical document collector and receives a plurality of clinical documents or clinical information from the clinical document collector. The clinical document collector is configured by instructions to execute a plurality of operations in an order to retrieve the plurality of clinical documents or clinical information, wherein each operation receive the task from the clinical data connector; retrieves the plurality of clinical documents or clinical information from the plurality of databases; and transmits the plurality of clinical documents or clinical information to the clinical data connector.

In some embodiments, the request for the clinical documents or clinical information is received from a customer portal application, wherein the customer portal application is configured by instructions to present and receive, via a graphical user interface, an executed general consent form from the patient.

In some embodiments, the request comprises an executed general consent form from the patient.

In some embodiments, the request from the customer portal application is through a SMART on FHIR API.

In some embodiments, the clinical document collector is configured by instructions to search a health exchange system for an available patient access list, wherein the available patient access list is used to create the task for the clinical document collector.

In some embodiments, the clinical data connector is configured to execute a state machine to track the retrieval of the plurality of clinical documents or clinical information.

In some embodiments, the clinical data connector is configured to poll the clinical document collector for a status request of each task, and wherein the reply of the status request include a link to a document store.

In some embodiments, the clinical data connector is configured to request the clinical document collector to request for the clinical documents or clinical information of the patient from the document store by sending a document request the document store using the link.

In some embodiments, the clinical data connector is configured to receive the request to initiate the operation for the retrieval of the clinical documents or clinical information of the patient from a third-party website or portal.

In another aspect, a method is disclosed comprising (a) receiving at a clinical data connector a request for clinical documents or clinical information of a patient, the request having (i) a plurality of patient identifier, (ii) a plurality of patient demographic identifier, and (iii) a purpose of use code for the request, (b) verifying the request and match a patient in a database using (i) and (ii); c) retrieving a plurality of clinical documents or clinical information; and (d) transmitting the plurality of clinical documents or clinical information a requester or designated third party receiver. The clinical data connector is configured by instructions to execute a plurality of operations in an order to retrieve the plurality of clinical documents or clinical information, wherein each operation creates a task for the clinical document collector and receives a plurality of clinical documents or clinical information from the clinical document collector. The clinical document collector is configured by instructions to execute a plurality of operations in an order to retrieve the plurality of clinical documents or clinical information, wherein each operation receive the task from the clinical data connector; retrieves the plurality of clinical documents or clinical information from the plurality of databases; and transmits the plurality of clinical documents or clinical information to the clinical data connector.

In some embodiments, the request for the clinical documents or clinical information is received from a customer portal application, wherein the customer portal application is configured by instructions to present and receive, via a graphical user interface, an executed general consent form from the patient.

Additional advantages of the invention will be set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate systems and methods for updating and validating medical records. Together with the description, the figures further serve to explain the principles of the systems and method described herein and thereby enable a person skilled in the pertinent art to make and use the systems and methods for updating and validating medical records.

FIG. 1 is an example environment for collecting and providing health-related information of a patient’s medical record or information from a plurality of health-service providers using a clinical data connector and clinical document collector;

FIG. 2 is a sequence diagram showing the operation of the registration between the platform and the web application or web service and subsequent data retrieval using the clinical data connector and clinical document collector;

FIG. 3 shows a detailed example implementation of the documents request and collection operation shown in FIG. 2 by the clinical data connector and clinical document collector;

FIG. 4 is a diagram showing an example state machine of the clinical data connector;

FIG. 5 shows an example process between the clinical document collector and a health exchange to query and retrieve clinical documents, e.g., based on the patient access implementation guide;

FIG. 6 shows a clinical document collector pipeline to locate and retrieve data for each patient presented in an incoming FHIR task;

FIG. 7 shows an FHIR API transaction for submitting a request to retrieve documents, monitoring the progress of the request, and downloading the documents retrieved in fulfillment of that request; and

FIG. 8 shows an exemplary computing environment in which example embodiments and aspects may be implemented.

### DETAILED DESCRIPTION

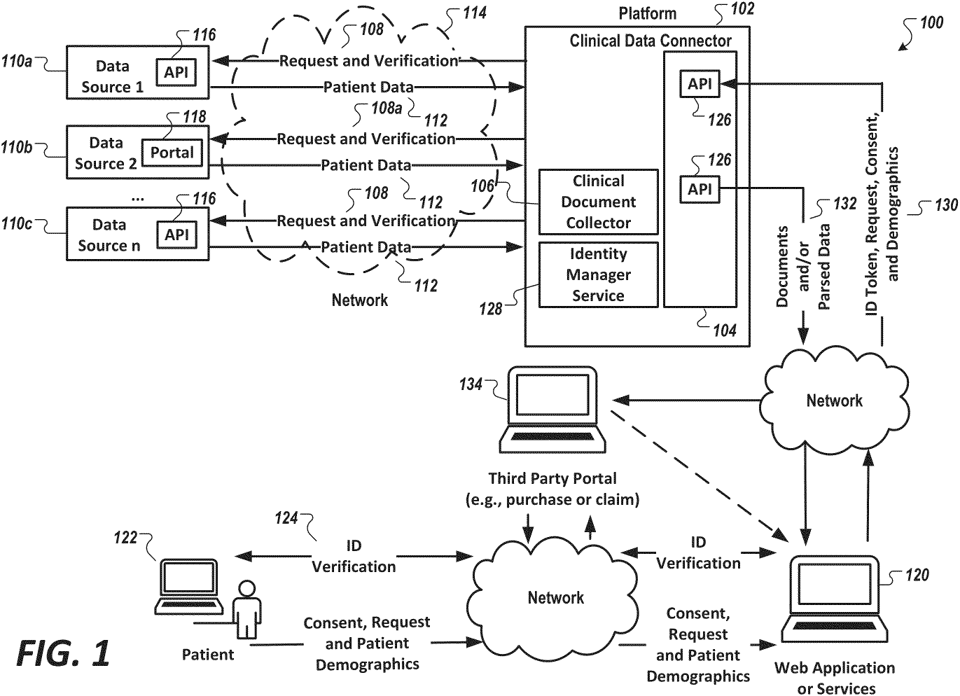

FIG. 1 shows an example environment 100 for collecting and providing health-related information of a patient’s medical record or other information using a clinical data connector 104 and clinical document collector 106. The clinical data connector 104 and clinical document collector 106 can collect records from a plurality of health-service providers using a single authorization release provided by the patient and without the patient having to provide the list of providers to the connector. Depending on the embodiment, the records in a records database may include medical records. Other types of records may be supported.

To facilitate the collecting and providing of the health-related information, the environment 100 includes a platform 102 that includes the clinical data connector 104 and the clinical document collector 106. In the example shown in FIG. 1, platform 102 can send requests and verification information 108 to a plurality of health-service providers 110 (shown as “Data Source 1” 110a, “Data Source 2” 110b, and “Data Source n” 110c) to retrieve records 112 (shown as “Patient Data” 112). The data sources (e.g., 110a, 110b, 110c) may be a part of the same platform 102 or may be controlled by a separate external entity. The records 112 are requested through a network 114. In some embodiments, the network 114 includes a local area network as well as a wide-area network.

In some embodiments, the data sources (e.g., 110a, 110b, 110c) may employ application-specific interfaces 116 that are configured to service a request (e.g., 108) and to transfer of the data 112 to the requester or provided receiver. In some embodiments, data source 110 may provide a portal 118 for the servicing of a request 108 (shown as 108a).

The platform 102 can operate with a web application or service 120 shown executing on a computing device in FIG. 1. The web application or service 120 is configured to receive consent from the patient for their medical records in an authorization release. The web application or service 120 can also receive purpose-of-use information for the consent. The consent and purpose of the consent can be transmitted from the web application or service 120 to the platform 102 that can retrieve and aggregate the patient records from the plurality of data sources 110. In the example shown in FIG. 1, the web application or web service 120 is configured (i) to present a general consent form to the patient through a customer portal curated by the web application or web service 120 and (ii) to receive the authorization information from the patient, e.g., as an electronic signature and/or selection of an element in the form. The web application or web service 120 also receives identifier information from a patient through a terminal 122 and performs verification 124 of the identity of the patient using the provided information.

Individuals (e.g., patients) can enter the web application or services through a terminal 122, which launches an ID proofing process. The verification may employ the NIST IAL level 2 verification process. IAL2 requires identity proofing that can require a government-issued identification (e.g., driver’s licenses information) in which the issuing entity had employed two pieces of superior or strong evidence to issue (e.g., government document, comparison to biometric).

The web application or web service 120 can generate an identity token and connect to the platform 102 through to an API 126 (e.g., SMART on FHIR API) to share the identity token and verify that the individual was proofed. The identity token can include specific patient demographic data (e.g., name, date of birth, home address, phone number, email address, etc.). The platform 102 can extract the demographic information from the token and send it to an identity manager service 128, which can match the patient to one or more local and/or global database(s) (e.g., a health data exchange, e.g., the CommonWell Health Alliance) to locate the patient’s data. The health data exchange may include additional functions in a suite of interoperability services (e.g., with APIs that can embed directly into a given application).

Once data are found in the local and/or global database(s), clinical document collector 106 can generate a request for the patient data from the data sources (e.g., 110a, 110b, 110c). In some embodiments, the request is in the form of a consolidated-clinical document architecture (C-CDA) compliant message or document. The C-CDA is a standard having common architecture, coding, semantic framework, and markup language for the creation of electronic clinical documents. The records 112, e.g., as C-CDA documents, are then returned to the platform 102. The C-CDA record (e.g., 112) may be parsed using FHIR API. Clinical document collector 106 can return a set of full C-CDAs to the platform 102 and the clinical data connector 104 can parse the C-CDA documents and return individual FHIR resources. The APIs (e.g., 116, 126) may employ SMART and FHIR, e.g., managed by HL7.

Indeed, the entire process is electronic. Individuals (e.g., patients) do not have to identify providers in advance or remember portal credentials from a provider. The process can be made much more user-friendly and ensure that all of a patient’s data is pulled back and parsed into structured data within the customer’s system rather than just viewing copies.

### Third-Party Portal

In an example implementation, the individual (e.g., patient) can access the web application or service 120 directly. The web application or service 120 is configured to receive information about the recipient of the record from the patient.

In another example implementation, the individual (e.g., patient) can access the web application or service 120 by being directed there through a third-party portal 134. The third-party portal 134 can be a company’s website that sells health-related products and services of that company, e.g., life insurance, disability insurance, supplemental, secondary life insurance, and the like. In some embodiments, the third-party portal 134 can be a medical insurance provider’s website or claim processor to process a claim, e.g., medical insurance claim, disability insurance claim, worker compensation claim, bodily injury insurance claim, medical, legal damage claim, and the like.

When accessing a third-party portal 134, upon purchasing or providing information for the purchase of a health-related service or to process a claim provided by the company, the third-party portal 134 can direct the individual (e.g., patient) to web application or web service 120, e.g., as an external internet link. In other embodiments, the third-party portal 134 can execute a client service of the web application or web service 120, which provides the data to the web application or web service 120. In yet other embodiments, the third-party portal 134 can execute a compatible service to acquire the data from the individual (e.g., patient) and then provide the acquired data to the web application or web service 120. In yet other embodiments, the third-party portal 134 can execute a compatible service to acquire the data from the individual (e.g., patient) and then provide the acquired data to the clinical data connector 104.

### Example Implementation Using SMART on FHIR

In one example, the platform 102 may execute a SMART on FHIR application to which an individual/patient can validate their identity. The SMART on FHIR application can employ a third-party identity proofing service. SMART is an open-source, standards-based API that offers secure and universal access to EHRs. The SMART platform builds on the existing FHIR standard, hence known as “SMART on FHIR.” The application will then use the identity-proofed unique identifier to register the person with a connector FHIR server (e.g., 102). This, in turn, triggers an activity in the connector server (e.g., 102) to locate clinical documents belonging to that person and cache the results in a local data store.

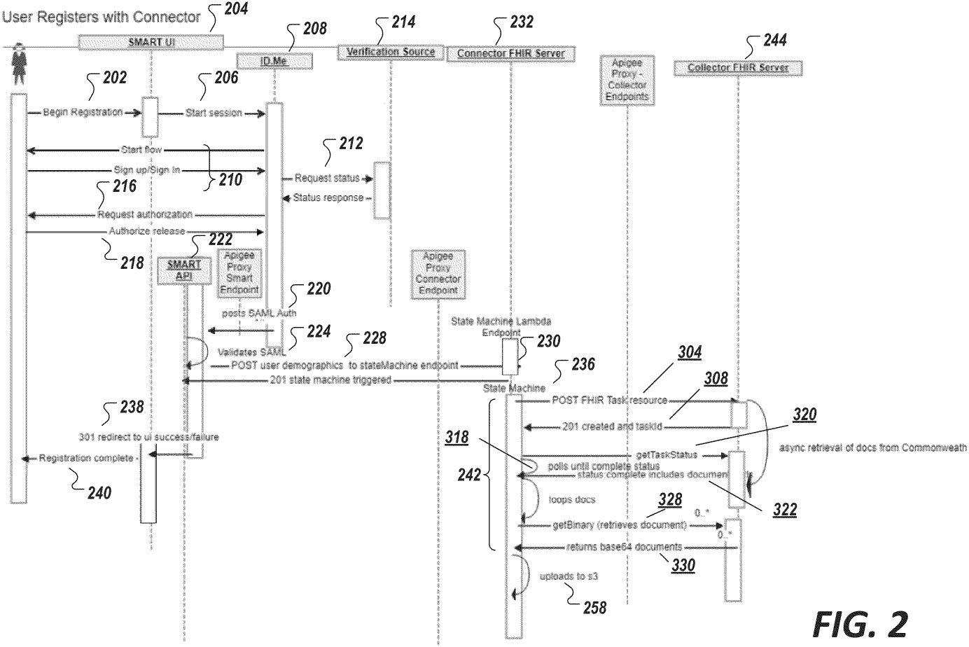

FIG. 2 is a sequence diagram showing the operation of the registration between the platform (e.g., 102) and the web application or web service 120 and subsequent data retrieval. As shown in the example of FIG. 2, a patient (show as “user”) can begin a registration (202) in a SMART user interface 204 (shown as “SMART UI” 204). Upon receiving an access request, the SMART UI 204 initiates (206) a session (208) for the patient (shown as “ID.Me” 208). The session 208 starts (210) a new flow and provides for the patient to sign up or to sign in into an account. The session 208 then authenticates or creates an authentication session 212 with a verification source manager 214 (shown as “Verification Source” 214). Following the authentication session 212, the session 208 sends (216) a request for HIPPA-related authorization to the user interface and the authorization release is then executed by the patient and transmitted (218) to the session 208.

Indeed, the request for HIPPA-related authorization includes a purpose-of-use indication and the user signature. This single HIPPA-related authorization release can be employed to generate a plurality of authorization releases with a plurality of data sources (e.g., 110a, 110b, 110c). In some embodiments, multiple HIPPA-related authorization releases may be presented and received, e.g., for each type of purpose of use.

Session 208 then transmits (220) via a POST message comprising a request for “SAML” authentication to a SMART-based API (222). Security Assertion Markup Language, or “SAML,” is a standardized protocol for an external application and/or service to determine a user is who they say they are. SAML employs single sign-on (SSO) technology to authenticate a user once and then communicate that authentication to multiple applications. The SMART-API 222 then performs validation (224) with the patient through the SMART UI (204) via SSO authentication. The patient also provides his or her demographic information through the SMART API 222 of the SMART UI 204 to send (228) a POST message comprising the user-provided demographic information to a state machine endpoint (230) of the connector FHIR server (232). Demographic information can include any identifying information about the person, such as name, DOB, address, phone, email, etc., that are available in a medical database to which the medical database can be queried to determine if a patient is present in that database.

Following an authentication session, the session 208 then initiates a new session for a state machine 236 at the connector FHIR server (232). The SMART API 222 then directs (238) the smart UI (204) to present a successful or failed session, and registration completes (240).

Upon initiating a new state machine session, the state machine (236) of the connector FHIR server (232) creates a new set of FHIR tasks to request documents from the collector service 244 (shown as “Collector FHIR Server”) collectively shown as 242.

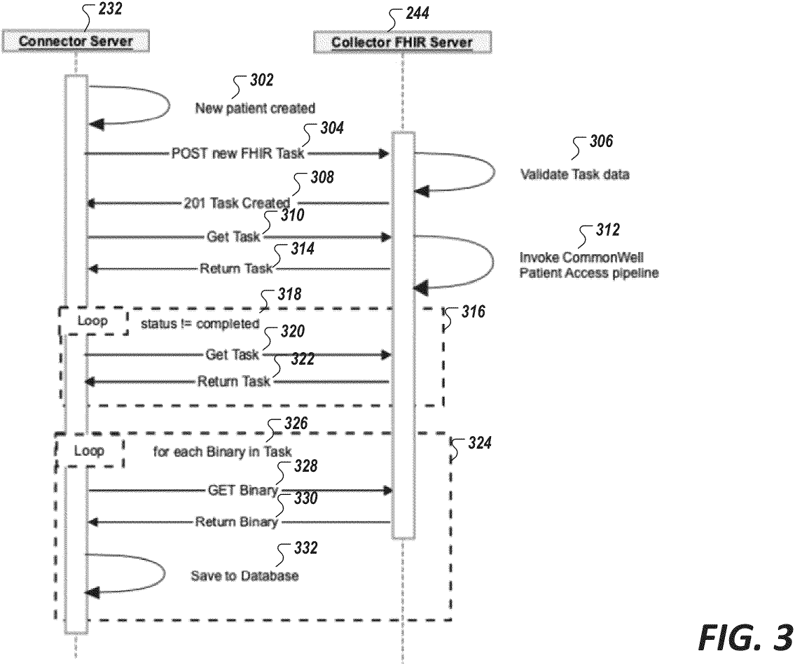

FIG. 3 shows a detailed example implementation of the documents request and collection operation shown in FIG. 2 by the connector 232 and collector service 244. Upon a new session being generated for a new patient (302), connector 232 first generates an FHIR task resource session (304) and sends (304) the FHIR task resource session to the collector of the FHIR server (244). The collector of the FHIR server (244) then validates the data contained in the incoming task to confirm it contains the required patient demographics and identity proofing. The validation may be performed by a NIST-compliant identity validation service. After validation (306), collector 244 assigns a globally unique identifier to the task resource and changes the status property to “Accepted.” Connector 232 saves the task resource to a durable data store and, the FHIR server sends (308) back an HTTP-201 created response and the updated task resource. The task resource is associated with a taskld (see FIG. 2) and is associated with the state machine for the retrieval of a list of available patient access from a health data exchange database. An available patient access refers to the presence of (i) an account or (ii) a record that is maintained by a provider for a given patient.

The connector (232) then sends (310) a request to determine, e.g., from the health data exchange database, the number of data sources associated with the patient using the provided patient demographic data. Collector 244 invokes (312) a health data exchange patient-access pipeline (e.g., CommonWell patient-access pipeline) and returns (314) the list of available patient access as tasks to connector 232. One or more pipeline processors perform a series of activities to locate and retrieve clinical data for the specified patient. As events occur during this processing, the connector (232) updates the data in the task resource for that patient to reflect the current state of processing, including events listed in Table 1.

TABLE 1

Events
Connector 232 initiates the processing of an accepted task and transitions the status to “In-Progress.”
Connector 232 determines a non-fatal error occurring and add FHIR OperationOutcome entry to the task.
Connector 232 determines a fatal error occurring and transition status to “Failed” and adds the FHIR operation outcome entry to the task.
Connector 232 determines the task is deleted by the data consumer and transitions the status to “Cancelled.”
Connector 232 determines documents are collected from the data provider and adds an output entry to the Task with a URL to a binary document.
Connector 232 determines the system has completed the processing of the task and transitions the status to “Completed.”

Because the API design provides a mechanism for the data consumers to retrieve the task from connector 232 at any time during and after processing (e.g., GET https://connector.chc.com/Task/15), the connector 232 may be obligated to return the current status and any additional information relevant to the processing of the request in the body of the Task resource that it returns in response to the request (e.g., errors and documents).

With the list of available patient access, the state machine 232 can initiate a set of asynchronous retrievals of documents from the data source databases (e.g., associated with 110a, 110b, 110c). In the example shown in FIG. 3, the connector server 232 execute an internal loop 318, in which each loop executes a request of the current status 320 (shown as GET task 320) that is sent to collector 244. The internal loop 318 ends when a status variable is set as completed. The task status request 320 may include a use field (e.g., official or not official) (e.g., line 10), a system field (e.g., line 11), a patient name field (e.g., lines 15-19), a patient gender field (e.g., lines 23-24), a patient address field (e.g., lines 24-31), a patient DOB field (e.g., line 34), an intent field for the request (e.g., comprising an indication of the request being an order, a reason code, a definition for the reason code, etc.) (e.g., lines 37-42), a current status field (e.g., lines 46-48), a restriction period field (e.g., lines 51-53), an execution period (e.g., lines 52-53), a service date range field (e.g., lines 63-70), among others.

Table 2 shows an example task status request 320. The example is shown in JSON and includes a reference to the line number.

TABLE 2

| | |
|---|---|
| Line Number | Code |
| 1 | { |
| | “resourceType”: “Task”, |
| | “id”: “123”, |
| | “contained”: [ |
| 5 | { |
| | “resourceType”: “Patient”, |
| | “id”: “p1”, |
| | “identifier”: [ |
| | { |
| 10 | “use”: “official”, |
| | “system”: “urn:oid:1.3.6.1.4.1.43927”, |
| | “value”: “12345” |
| | } |
| | ], |
| 15 | “name”: [ |
| | { |
| | “family”: “Public”, |
| | “given”: [ |
| | “John” |
| 20 | ] |
| | } |
| | ], |
| | “gender”: “male”, |
| | “address”: [ |
| 25 | { |
| | “line”: [ |
| | “100 Main St” |
| | ], |
| | “city”: “Metropolis”, |
| 30 | “state”: “II”, |
| | “postalCode”: “44130” |
| | } |
| | ], |
| | “birthDate”: “1956-05-27” |
| 35 | } |
| | ], |
| | “intent”: “order”, |
| | “reasonCode”: { |
| | “coding”: [ |
| 40 | { |
| | “system”: “http://healthit.gov/nhin/purposeofuse”, |
| | “code”: “REQUEST” |
| | } |
| | ] |
| 45 | }, |
| | “status”: “in-progress”, |
| | “focus”: { |
| | “reference”: “#p1” |
| | }, |
| 50 | “restriction”: { |
| | “period”: { |
| | “end”: “2020-10-02T00:00:00+08:00” |
| | } |
| | }, |
| 55 | “executionPeriod”: { |
| | “start”: “2020-10-01T09:45:00+08:00” |
| | }, |
| | “input”: [ |
| | { |
| 60 | “type”: { |
| | “coding”: [ |
| | { |
| | “system”: “fhir.chc.input”, |
| | “code”: “serviceDateRange” |
| 65 | } |
| | ] |
| | }, |
| | “valuePeriod”: { |
| | “start”: “2020-01-30”, |
| 70 | “end”: “2020-02-10” |
| | } |
| | } |
| | ] |
| | } |

In reply to each GET task status request 320, the connector FHIR server (232) attempts to retrieve records from each of the data sources (e.g., 110a, 110b, 110c) and sends a current status of that request to the data sources as a RETURN task reply 322.

Once the document retrieval task is complete, the final status of the task includes a link to a document bundle and the data source identifier/address. Table 3 shows an example output of the final task status request.

TABLE 3

| | |
|---|---|
| Line Number | Code |
| 1 | “output”: [ |
| | { |
| | “type”: { |
| | “coding”: [ |
| 5 | { |
| | “system”: “fhir.chc.output”, |
| | “code”: “document” |
| | } |
| | ] |
| 10 | }, |
| | “valueReference”: { |
| | “reference”: “Binary/456” |
| | } |

With the list of links to the records, the state machine 232 can initiate a set of asynchronous retrievals of documents from the data source databases (e.g., associated with 110a, 110b, 110c). In the example shown in FIG. 3, the connector server 232 executes an internal loop 326 in which each loop executes a request of the binary 328 (shown as GET task 328) that is sent to the collector 244. The connector FHIR server (232) receives a return binary 330 from the collector of the FHIR server (244) and saves the binary to its database 332. Connector 232 may parse the retrieved documents to return (256) FHIR compliant documents to the connector FHIR server (232). The connector FHIR server (232) then provides (258) the FHIR documents to the third-party system. In the example, the FHIR documents are shown housed in Amazon Simple Storage Services (shown as “S3”).

Table 4 shows an example request for binary 328. Table 5 shows an example reply for binary 330.

TABLE 4

| | |
|---|---|
| Line Number | Code |
| 1 | GET https://fhir.chc.com/Binary/456 HTTP/1.1 |
| | Host: fhir.chc.com |
| | Authorization: Bearer mF_9.B5f-4.1JqM |

TABLE 5

| Line Number | Code |
|---|---|
| 1 | GET https://fhir.chc.com/Binary/456 HTTP/1.1 |
| | Host: fhir.chc.com |
| | Authorization: Bearer mF_9.B5f-4.1JqM |

### Connector State Machine

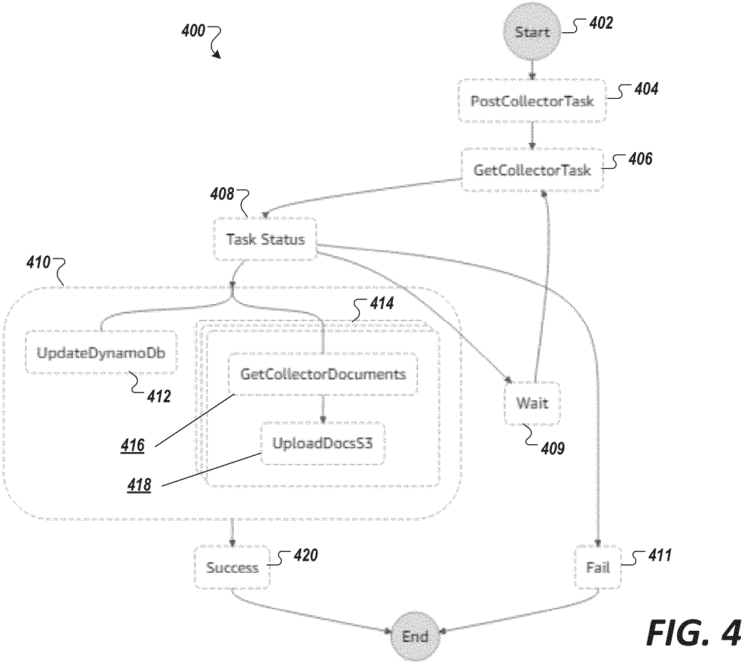

FIG. 4 is a diagram showing a Connector State Machine 400, e.g., for connector 232). The state machine 400 initiates at a start state 402. The state machine 400 then transitions to a PostCollectorTask state 404 to perform operation 304 (e.g., that generates a FHIR task resource session and sends the FHIR task resource session to the collector of the FHIR server). Once completed, the state machine 400 transitions to a GetCollectorTask state 406 to perform operation 308 (e.g., that receives an HTTP-201 created response and the updated task resource). The state machine 400 then transitions to a Task Status state 408 (e.g., to execute the internal loop 318 in which each loop executes a request of the current status 320). The status request can return a competed status, a wait status, or a failed operation status, and the state machine 400 can transition to retrieve document states 410, a wait state 409, or a fail state 411.

In the document retrieval state 410, the state machine 400 transitions to an update database state 412 if there is no record to retrieve based on the task status. The state machine 400 initiates a set of sub-states 414 that each enters a collector document retrieval state 416 and an upload document state 418 to upload the retrieved files to a third-party system. Once all the documents are retrieved, the state machine 400 enters the success state 420.

### Health Data Exchange Patient Access

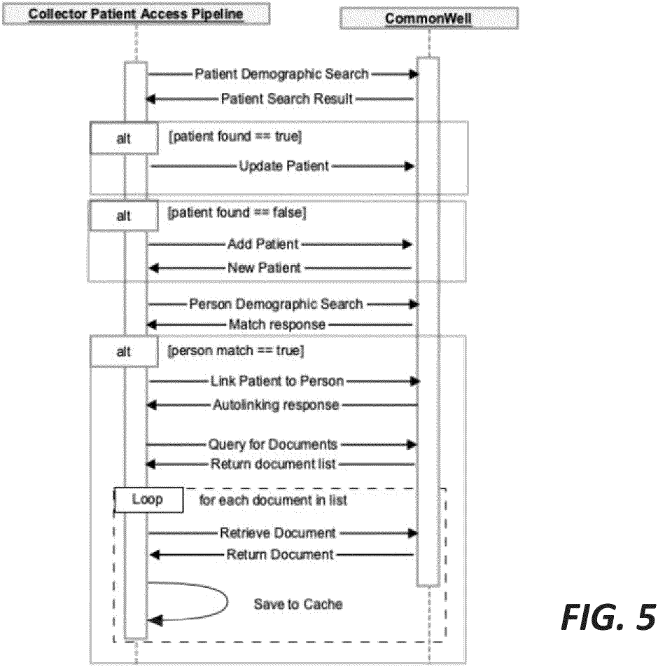

FIG. 5 shows an example process between the collector and a health exchange (shown as “CommonWell”) to query and retrieve clinical documents based on the patient access implementation guide.

Tables 6 and 7 respectively show a JSON message for the required data and for a request.

In Table 6, the identifier array may contain other identifiers but MUST include an identifier that has been issued by a NIST compliant identity validation service. In Table 7, the First Name is provided as the first element in the “given” array. The Middle Name(s) will be the next elements in the “given” array. Each given name will be in its own element. Additional data may be transmitted, including the Patient Last Name, the Patient Date of Birth, the Patient Postal Code, and the Service Date Range.

TABLE 6

```
{
    “resourceType”: “Task”,
    “contained”: [
    {
          “resourceType”: “Patient”,
          “identifier”: [
          {
                “use”: “official”,
                “system”: “urn:oid:1.3.6.1.4.1.43927”,
                “value”: “12345”
          }
    ]
    ...
}
```

TABLE 7

```
{
    “resourceType”: “Task”,
    “contained”: [
    {
          “resourceType”: “Patient”,
          “name”: [
          {
                “given”: [
                “John”,
                “Brad”
                ]
          }
          ],
    ] ...
}
```

### Connector Event Pipeline

The Connector application includes event-driven processing pipelines. This architectural pattern provides for separation of concerns, component isolation, and for extending and updating the system to support new types of data sources and/or consumers, post-processing of CDA documents, as well as adjacent use cases for reporting and analytics.

The publicly accessible API for the Connector services supports the asynchronous, task-based workflow. In considering a technical approach, the design considers using the standard FHIR resource model for implementing an API.

In some embodiments, the connector is implemented according to a Da Vinci Clinical Data Exchange (CDex) Implementation Guide (IG). This design has considered the Da Vinci Clinical Data Exchange (CDex) implementation guide published by HL7, providing guidance on exchange methods, payloads, and search criteria for the exchange of clinical information between provider and other providers and/or payers.

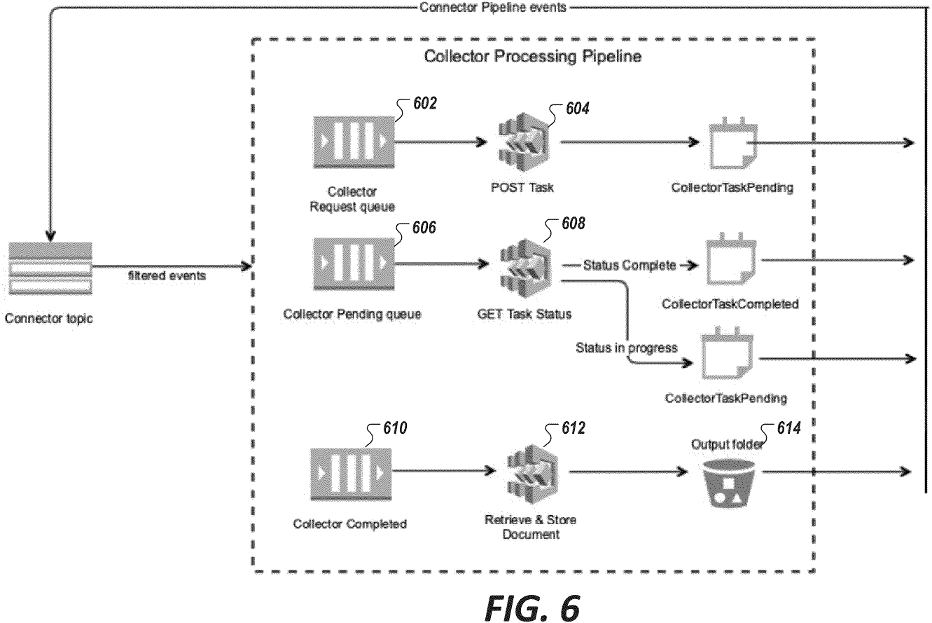

FIG. 6 shows a collector pipeline of a process to locate and retrieve data for each patient presented in an incoming FHIR Task. For simplicity, the diagram omits a durable or non-durable cache for context data, which will be described in detail below. This pipeline can be realized in an AWS Step Function. Each of the queues in the pipelines receives a filtered event from the Connector topic.

Collector Request queue (602): an SQS queue receiving filtered TaskAccepted events (the triggering event for every data retrieval pipeline).

POST Task function (604): a sequence of functions responsible for retrieving a patient record from the Patient data store, extracting the NPI from the record and using a health data exchange REST-based Home Community ID lookup service (e.g., CommonWell’s REST ID lookup service) to retrieve a list of associated Home Community Identifiers. On success, publishes a CollectorTaskPending event.

Collector Pending queue (606): an SQS queue receiving filtered CollectorTaskPending events.

Get Task Status function (608): invokes a GET on the Task against the Collector FHIR API to retrieve the current version of the Task. The function then interrogates the status property: if the status is still in-progress, the function publishes a CollectorTaskPending event; if complete, the function publishes a CollectorTaskCompleted event.

Collector Completed queue (610): an SQS queue receiving filtered CollectorTaskCompleted events.

RetrieveAndStore function (612): retrieves the document from Collector and stores the result to the designated output folder.

Output folder (614): an S3 bucket to hold the retrieved documents.

The pipeline can back up events from the Connector topic to an Amazon S3 bucket, e.g., using an Amazon Kinesis Data Firehose stream.

### Example API

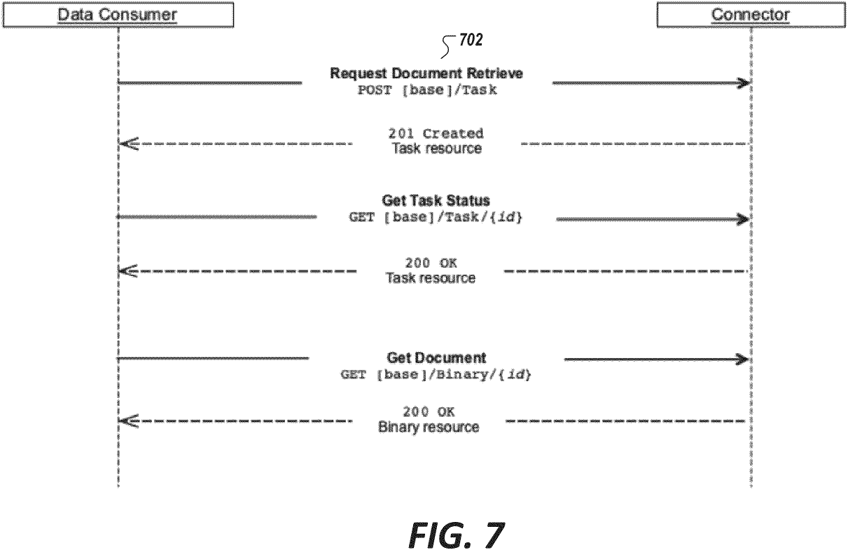

FIG. 7 shows a FHIR API transaction for submitting a request to retrieve documents, monitoring the progress of the request, and downloading the documents retrieved in fulfillment of that request.

POST [base/Task] (702): Initiates a request for retrieval of clinical documents for an identity-proofed individual. Table 8 shows information included in the request.

GET [base(see page 8)1/Binary/{id4}: Returns a retrieved document from the server using the link provided in the Task resource output property.

TABLE 8

| | |
|---|---|
| Field | Description |
| focus | a reference to a contained Patient resource |
| reasonCode | the purpose of use (use http://healthit.gov/nhin/purposeofuse2) will always be “REQUEST” |
| patient | the contained patient resource, including an identifier (the ID.Me identifier, the registered system OID is 1.3.6.1.4.1.43927), family name, given name, gender, birthdate, address, and postal code |
| restriction.period | date and time by which document retrieval MUST be completed input (input types defined in the fhir.chc.input value set) |
| serviceDateRange | the date range for document query (value is a Period) |

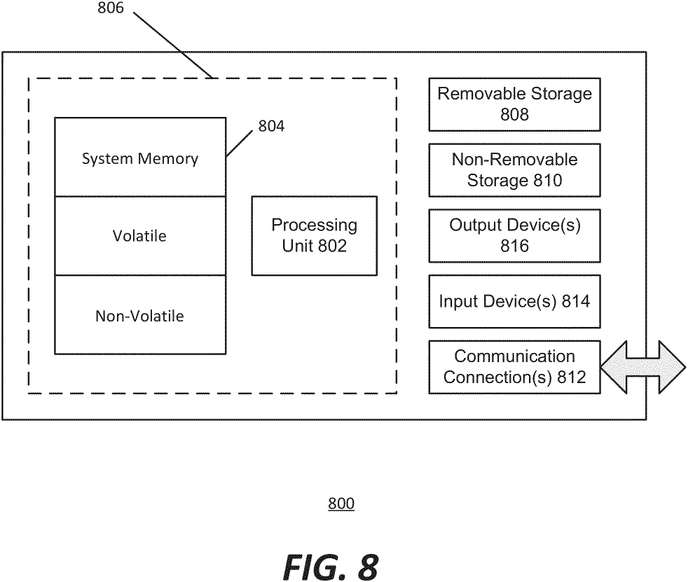

FIG. 8 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media, including memory storage devices.

With reference to FIG. 8, an exemplary system for implementing aspects described herein includes a computing device, such as computing device 800. In its most basic configuration, computing device 800 typically includes at least one processing unit 802, and memory 804. Depending on the exact configuration and type of computing device, memory 804 may be volatile (such as random access memory (RAM)), non-volatile (such as read-only memory (ROM), flash memory, etc.), or some combination of the two. This most basic configuration is illustrated in FIG. 8 by dashed line 806.

Computing device 800 may have additional features/functionality. For example, computing device 800 may include additional storage (removable and/or non-removable) including, but not limited to, magnetic or optical disks or tape. Such additional storage is illustrated in FIG. 8 by removable storage 808 and non-removable storage 810.

Computing device 800 typically includes a variety of computer-readable media. Computer-readable media can be any available media that can be accessed by the device 800 and includes both volatile and non-volatile media, removable and non-removable media.

Computer storage media include volatile and non-volatile, and removable and non-removable media implemented in any method or technology for storage of information such as computer-readable instructions, data structures, program modules or other data. Memory 804, removable storage 808, and non-removable storage 810 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 800. Any such computer storage media may be part of computing device 800.

Computing device 800 may contain communication connection(s) 812 that allow the device to communicate with other devices. Computing device 800 may also have input device(s) 814 such as a keyboard, mouse, pen, voice input device, touch input device, etc. Output device(s) 816 such as a display, speakers, printer, etc., may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still, further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system comprising:
at least one computing device having instructions stored thereon that execute a clinical data connector and a clinical document collector configured to:
(a) receive, from a requester, a request for one or more clinical documents or clinical information of a patient, the request having (i) a patient identifier, (ii) patient demographic information, and (iii) a purpose of use code for the request,
(b) verify the request and match a patient in at least one database using (i) and (ii);
(c) retrieve the clinical documents or clinical information from the at least one database; and
(d) transmit the clinical documents or clinical information to the requester or a designated third party receiver;
wherein the clinical data connector is configured by instructions to execute a plurality of operations in an order to retrieve the clinical documents or clinical information, wherein each operation creates a task for the clinical document collector and receives at least one clinical document or clinical information from the clinical document collector; and
wherein the clinical document collector is configured by instructions to execute a plurality of operations in an order to retrieve the clinical documents or clinical information, wherein each operation:
receives the task from the clinical data connector;
retrieves the clinical documents or clinical information from the at least one database; and
transmits the clinical documents or clinical information to the clinical data connector.

2. The system of claim 1, wherein the request for the clinical documents or clinical information is received from a customer portal application, wherein the customer portal application is configured by instructions to present and receive, via a graphical user interface, an executed general consent form from the patient.

3. The system of claim 1, wherein the request comprises an executed general consent form from the patient.

4. The system of claim 2, wherein the request from the customer portal application is through a SMART on FHIR API.

5. The system of claim 1, wherein the clinical document collector is configured by instructions to search a health exchange system for an available patient access list, wherein the available patient access list is used to create the task for the clinical document collector.

6. The system of claim 5, wherein the clinical data connector is configured to execute a state machine to track the retrieval of the clinical documents or clinical information.

7. The system of claim 5, wherein the clinical data connector is configured to poll the clinical document collector for a status of each task, and wherein the reply of the status request includes a link to a document store.

8. The system of claim 7, wherein the clinical data connector is configured to request the clinical documents or clinical information of the patient from the document store by sending a document request to the document store using the link.

9. The system of claim 1, wherein the clinical data connector is configured to receive the request to initiate the operation for the retrieval of the clinical documents or clinical information of the patient from a third-party website or portal.

10. The system of claim 9, wherein the clinical data connector and/or clinical document collector is configured by instructions to parse through the retrieved clinical documents or clinical information to populate a specific set of requested medical information in the third-party website or portal.

11. A non-transitory computer readable medium having instructions stored thereon that executes a clinical data connector and a clinical document collector configured to
(a) receive, from a requester, a request for one or more clinical documents or clinical information of a patient, the request having (i) a patient identifier, (ii) a patient demographic identifier, and (iii) a purpose of use code for the request,
(b) verify the request and match a patient in at least one database using (i) and (ii);
(c) retrieve the clinical documents or clinical information from the at least one database; and
(d) transmit the clinical documents or clinical information to the requester or a designated third party receiver;
wherein the clinical data connector is configured by instructions to execute a plurality of operations in an order to retrieve the clinical documents or clinical information, wherein each operation creates a task for the clinical document collector and receives at least one clinical document or clinical information from the clinical document collector; and
wherein the clinical document collector is configured by instructions to execute a plurality of operations in an order to retrieve the clinical documents or clinical information, wherein each operation:
receives the task from the clinical data connector;
retrieves the clinical documents or clinical information from the at least one database; and
transmits the clinical documents or clinical information to the clinical data connector.

12. The non-transitory computer readable medium of claim 11, wherein the request for the clinical documents or clinical information is received from a customer portal application, wherein the customer portal application is configured by instructions to present and receive, via a graphical user interface, an executed general consent form from the patient.

13. The non-transitory computer readable medium of claim 11, wherein the request comprises an executed general consent form from the patient.

14. The non-transitory computer readable medium of claim 12, wherein the request from the customer portal application is through a SMART on FHIR API.

15. The non-transitory computer readable medium of claim 11, wherein the clinical document collector is configured by instructions to search a health exchange system for an available patient access list, wherein the available patient access list is used to create the task for the clinical document collector.

16. The non-transitory computer readable medium of claim 14, wherein the clinical data connector is configured to execute a state machine to track the retrieval of the clinical documents or clinical information.

17. The non-transitory computer readable medium of claim 15, wherein the clinical data connector is configured to poll the clinical document collector for a status of each task, and wherein the reply of the status request include a link to a document store.

18. The system of claim 17, wherein the clinical data connector is configured to request the clinical documents or clinical information of the patient from the document store by sending a document request to the document store using the link.

19. The system of claim 11, wherein the clinical data connector is configured to receive the request to initiate the operation for the retrieval of the clinical documents or clinical information of the patient from a third-party website or portal.

20. A method comprising:
(a) receiving, from a requester, a request for one or more clinical documents or clinical information of a patient, the request having (i) a patient identifier, (ii) a patient demographic identifier, and (iii) a purpose of use code for the request,
(b) verifying the request and match a patient in at least one database using (i) and (ii);
(c) retrieving the clinical documents or clinical information from the at least one database; and
(d) transmitting the clinical documents or clinical information to the requester or a designated third party receiver;
wherein the clinical data connector is configured to execute a plurality of operations in an order to retrieve the clinical documents or clinical information, wherein each operation creates a task for a clinical document collector and receives at least one clinical document or clinical information from the clinical document collector; and
wherein the clinical document collector is configured to execute a plurality of operations in an order to retrieve the clinical documents or clinical information, wherein each operation receive the task from the clinical data connector; retrieves the plurality of clinical documents or clinical information from the plurality of databases; and transmits the plurality of clinical documents or clinical information to the clinical data connector.

21. The method of claim 20, wherein the request for the clinical documents or clinical information is received from a customer portal application, wherein the customer portal application is configured by instructions to present and receive, via a graphical user interface, an executed general consent form from the patient.
